(19) United States
(12) Patent Application Publication
(10) Pub. No.: US 2024/0251851 A1
(43) Pub. Date: Aug. 1, 2024

(54) Container for an Inhalation Device with at Least One Liquid Jet Device, Combination of at Least Two Containers and Method of Conveying Liquid to an Inhalation Device

(71) Applicant: JT International SA, Geneva (CH)

(72) Inventors: James Mott, San Diego, CA (US); Simon Dodd, Albany, OR (US); Bruce Eckard, San Diego, CA (US); Steve Burney, Escondido, CA (US); Ari Badal, Barcelona (ES)

(73) Assignee: JT International SA, Geneva (CH)

(21) Appl. No.: 18/289,714

(22) PCT Filed: May 4, 2022
(86) PCT No.: PCT/EP2022/061960

§ 371 Date: Nov. 6, 2023

(30) Foreign Application Priority Data
May 6, 2021 (EP) 21172475.2

(51) Int. Cl. A24F 40/42; A24F 40/10; A24F 40/44; A24F 40/48

(52) U.S. Cl. CPC A24F 40/42; A24F 40/10; A24F 40/44; A24F 40/48

(57) ABSTRACT

A container (30) for an inhalation device (10) has at least one liquid jet device (24), such as a micro heater or resistor, the container (30) being adapted to deliver liquid to the liquid jet device (24) and at least one capillary tube (42) extending from a delivery outlet, and at least one absorbing element (46), such aass a piece of foam oorr a wick, adapted to absorb liquid, the capillary tube (42) and/or the absorbing element ((4466)) extending substantially to aa bottom of the container (30) opposite the delivery outlet. Two such containers can be combined to form a cartridge insertable into the inhalation device, and the invention is also related to a method of conveying liquid to an inhalation device by means ooff a combination of a capillary tube and an absorbing element (FIG. 2).

### TECHNICAL FIELD

The invention relates to a container for an inhalation device with at least one liquid jet device, a combination of the two containers forming a cartridge and a method of conveying liquid to an inhalation device.

In recent years, inhalation devices such as e-cigarettes have become popular as a replacement for conventional smoking articles. In this context, a certain amount of liquid has to be stored in the inhalation device, preferably in a replaceable cartridge and has to be delivered appropriately to a heater so as to form an aerosol.

### PRIOR ART

US 2020 0308107 A1 is related to such a smoking article comprising a reservoir containing an aerosol precursor composition being in fluid communication with a microheater.

A similar vaporizer is derivable from U.S. Ser. No. 10/172,388 B2.

Finally, KR 101923047 B1 discloses an aerosol generating system having a container with a liquid and a capillary wick contacting the liquid.

### SUMMARY OF THE INVENTION

In view of the above, there remains a need for a container for an inhalation device with at least one liquid jet device, which can provide an appropriate flow of liquid to the liquid jet device, in particular independent from the orientation of the inhalation device.

This is achieved by means of the container in accordance with claim 1, which has at least one capillary tube extending from a delivery outlet and at least one absorbing element, the capillary tube and/or the absorbing element extending substantially to the bottom of the container opposite the delivery outlet.

In this manner, two opposing forces are taken into account and balanced in order to provide the appropriate flow of liquid, i.e. enough liquid to allow appropriate aerosol generation in any orientation of the device, and not too much liquid, in order to avoid “drooling” of liquid. This is essentially achieved by generating capillary forces in combination with gravity to create sufficient forces to pull or draw liquid from the reservoir to the liquid jet device, in particular a microheater or a resistor. At the same time, in any orientation of the device, enough negative pressure is created, which “sucks” the liquid into the container functioning as a reservoir in order to avoid too much liquid being expelled from the device. Moreover, the invention serves to provide fluid damping to the liquid delivery system, so that essentially all liquid in the reservoir can be delivered, before air enters the liquid path, which would cause a deprime.

In detail, the capillary tube, which can also be called a pickup tube or dip tube extends from the delivery outlet of the container and essentially draws liquid towards the delivery outlet, when the device is oriented with the delivery outlet essentially at the top. The absorbing element is combined with the capillary tube and provides enough back pressure to avoid too much liquid to be delivered to the liquid device, even when the device is oriented with the delivery outlet downwards. In addition, the absorbing element, which can for example be a piece of foam or a wick, absorbs a certain amount of liquid and retains this to provide a kind of damper, so that even in case of a rapid movement or “sloshing” of the container, air does not enter the liquid delivery system. Further, the liquid retained in the capillary tube and the absorbing element allows the device to function for some time, even when the delivery outlet is oriented downwards and the level of liquid is too low to allow the capillary tube or the absorbing element to contact the liquid. Finally, the described system can be produced in a cost-efficient manner and, as described, provides a reliable delivery of liquid to a liquid jet device in any orientation of the inhalation device.

The absorbing element can for example be inserted at the end of the capillary tube opposite the delivery outlet. As regards materials for the absorbing element, the wick can be formed of cigarette filter material. The liquid jet device as described herein essentially corresponds to an inkjet device commonly used in printers.

Preferred embodiments are described in the further claims.

As already indicated, as soon as about 30% or more of the liquid in the container has been used, there can be a situation, depending on the length of the combination of the capillary tube and the absorbing element, in which neither one of these contacts the liquid, when the device is held in an orientation with the delivery outlet downwards. Also in such a situation, it is desirable that the device continues to work for a certain period of time, and it is, therefore, preferred that a volume of the capillary tube corresponds to two or three puffs. In other words, even in the described orientation, the user can vape for two or three puffs, before the capillary tube is empty, and the orientation of the device has to be changed, in order to fill the capillary tube with liquid. In this context, a volume of one puff may correspond to a volume of at least approximately 5 μL (assuming that at least 5 mg of liquid is delivered per puff and that the density of the liquid is similar to that of water). Thus, a volume of two or three puffs may correspond to a volume of at least approximately 10 μL or 15 μL, respectively. It is preferred that a volume of the capillary tube corresponds to at least 10 μL or at least 15 μL.

As regards the inner diameter of the capillary tube in order to ensure the reliable delivery of liquid, an inner diameter of 1 to 3 mm has proven efficient. Beyond 3 mm and in particular towards 4 mm, capillary action cannot be ensured any longer, in particular when using liquids with a low surface tension. On the other hand, with an inner diameter below 1 mm, the inner volume of the capillary tube becomes very small, the flow rate decreases and the delivery of liquid is stopped too quickly.

As regards dimensions of the capillary tube and the absorbing element, any combinations are possible. For example, the capillary tube can be between 5 and 30 mm long, and the same applies to the length of the absorbing element. In other words, a very short capillary tube, for example having a length of only 5 mm, can be combined with a very long absorbing element, such as a wick, having a length of for example 3 cm. Such a combination has turned out to work as well as an absorbing element being only 3 mm long and inserted into a capillary tube being for example 3 cm long. Nevertheless, in view of a beneficial combination of total volume and delivered liquid, it is preferably that the capillary tube is longer than the absorbing element. In particular, a combination of an about 3 cm long capillary tube, having an inner diameter of up to 3 mm, with an absorbing element of only about 3 mm length inserted, is currently preferred. A comparably small absorbing element also has the advantage of keeping the retention force on the liquid low and keeping the flow rate high.

For example, if the capillary tube has an inner diameter of 1 mm, the capillary tube would need to have a length of at least 20 mm in order to hold a volume of at least 15 μL (corresponding to three puffs). On the other hand, if the capillary tube has an inner diameter of 2 mm, the capillary tube would need to have a length of at least 5 mm in order to hold a volume of at least 15 μL (corresponding to three puffs).

As already indicated, a particularly simple structure can be achieved, when the absorbing element is attached to and preferably inserted into a bottom side end of the capillary tube, in other words, the end opposite of the delivery outlet.

Good results have also been achieved with a connecting tube having an inner diameter at least as large as an outer diameter of the capillary tube and the absorbing element, the connecting tube being placed radially outwards the capillary tube and the absorbing element and connecting these.

Filling of the container can be made particularly easy, when the container has a cap, to which the capillary tube is attached. Thus, after filling the container it can be closed, and the capillary tube together with the absorbing element can be brought in place in a single step. The cap is preferably connected to the container by means of a thread.

As also indicated above, it provides benefit to the user, when the combination of the capillary tube and the absorbing element is adapted to deliver liquid to the delivery outlet, also when the delivery outlet is oriented vertically above the bottom of the container. For example, this can be achieved by the capillary tube having an inner diameter of 1-3 mm and a length of 5-30 mm, and the absorbing element having a length of 3-30 mm.

At the same time, it is preferred that liquid is retained, when the delivery outlet is oriented vertically below the bottom of the container, i.e. the device is held with the delivery outlet oriented downwards. For example, this can be achieved by the capillary tube having an inner diameter of 1-3 mm and a length of 5-30 mm, and the absorbing element having a length of 3-30 mm.

As regards the material of the capillary tube, Teflon, Polypropylene and Polyethylene have proven efficient and can also be combined for production of the capillary tube.

The desired capillary forces when using the mentioned materials can particularly be achieved, when the liquid is for example a polypropylene glycol/vegetable glycerine mixture or an oil-based perfume, which are common liquids for inhalation devices or aerosol generating devices.

In the configuration of the container described above, the absorbing element may be disposed within the container and/or the absorbing element and the capillary tube are disposed within a same compartment of the container.

In order to further increase the amount of liquid and the user benefit, at least two containers as described above can be combined so as to form a cartridge insertable into the inhalation device.

As can also be taken from the above, the invention advantageously combines at least one container or cartridge as previously described with an inhalation device having at least one liquid jet device, which is preferably in the form of an MEMS.

Correspondingly, the invention also provides a method of conveying liquid to an inhalation device by means of a combination of a capillary tube and an absorbing element, which is preferably effected from at least one replaceable container or cartridge. Any features described herein with reference to the container or cartridge can be applied to the method and vice versa.

### BRIEF DESCRIPTION OF THE DRAWINGS

Hereinafter, the invention is further described with reference to the drawings, in which:

FIG. 1 shows a perspective view of the inhalation device;

FIG. 2 shows a cut view of essential parts of the inhalation device of FIG. 1;

FIG. 3 shows two liquid jet devices in greater detail.

### DETAILED DESCRIPTION OF A PREFERRED EMBODIMENT

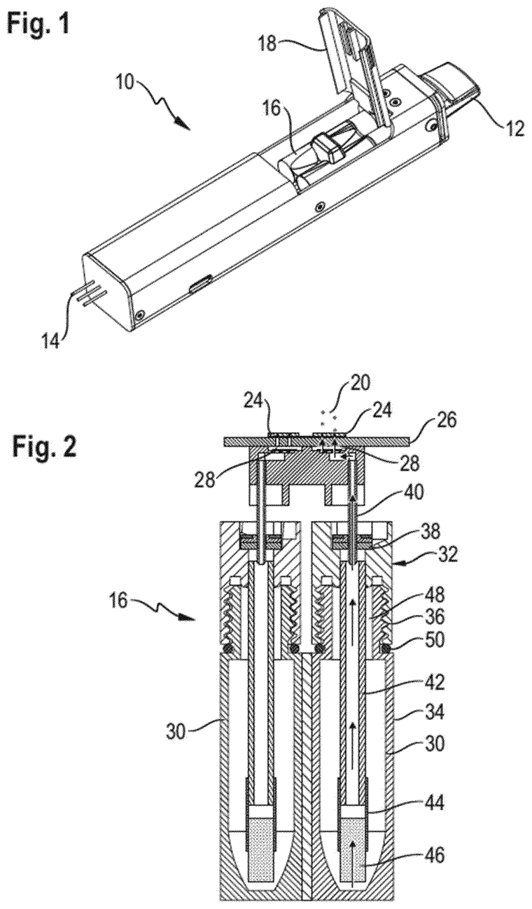

As can be taken from FIG. 1, the inhalation device 10 as described herein, is essentially elongate with a mouthpiece 12 at one end and charging contacts 14 at the other end. Any aerosol delivered to the mouthpiece 12 is produced from liquid contained in a cartridge 16, which is essentially accommodated in that half of the inhalation device 10, which is closer to the mouthpiece 12. In the case shown, the cartridge 16 is accommodated in a compartment which can be closed by a lid 18, and a longitudinal axis of the device 10 corresponds to a longitudinal axis of the cartridge 16 and containers forming same as described in more detail below.

As can better be seen in FIG. 2, aerosol 20 is produced by in the present case two liquid jet devices 24, which are provided on a PCA or daughter board 26 with a filter 28 between the liquid delivery system described in more detail below and each liquid jet device 24. As can be seen in the lower part of FIG. 2, the cartridge in this case contains two containers 30, which are substantially structured identically, so that only one of them will be described in more detail below. In an upper part of the container 30 it essentially has a cap 32, which can be screwed onto the reservoir part 34 by means of a thread 36. In the present case, the cap 32 has at least one septum 38, which can be punctured by a hollow needle 40 which is part of the inhalation device 10 in accordance with FIG. 2. Anything below needle 40 in FIG. 2 belongs to the cartridge 16, which is replaceable. In the embodiment shown, a capillary tube 42 is attached to the cap 32 and is adapted to deliver liquid towards the delivery outlet at the top of the cartridge 16 in accordance with the orientation of FIG. 2. The delivery direction of the liquid is indicated by arrows.

In the embodiment shown, a connecting tube 44 is attached outside the capillary tube 42 and an absorbing element 46 is attached inside the connecting tube 44. The capillary tube 42 and the absorbing element 46 are disposed within a same compartment of the container 30. In the case shown, there is a certain gap between the capillary tube 42 and the absorbing element 46, but this gap does not necessarily have to be present and can also be greater than shown. As can also be taken from FIG. 2, the absorbing element 46, such as a wick, extends substantially to the bottom of the container 30, so that in the orientation of FIG. 2, essentially all of the liquid contained can be drawn towards the delivery outlet. In the embodiment shown, both caps 32 are essentially circle cylindrical, and this also applies to the reservoir parts 34, which are connected with each other. The inner volume of the reservoir part 34 is also essentially circle cylindrical, in the embodiment shown with a cone shaped lower part. Further, in the embodiment shown, a suitable air path 48 is provided between the capillary tube 42 and the inner surface of the reservoir part 34 in the area of the thread 36 so as to allow air to enter the reservoir part and to avoid the build-up of excessive negative pressure. Further, in the embodiment shown, an O-ring 50 is provided between the reservoir part 34 and the cap 32 as a gasket.

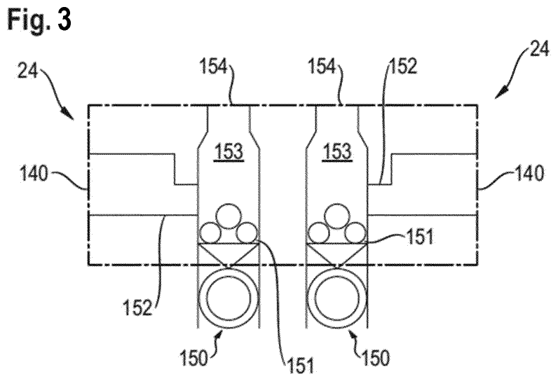

FIG. 3 represents in a cross-sectional diagram a portion of an example of an inhalation or aerosol-generation device.

The combination of two liquid jet devices 24 comprises here two microfluidic structures or MEMS dies 150.

Each MEMS die 150 has an upper surface or vaporization surface 151.

The liquid jet devices 24 are in fluid communication with two liquid channels 140 each of which is arranged to deliver the liquid aerosolizable material from the reservoir part 34. Each liquid channel 140 is connected to an MEMS die 150 through an inlet port 152. Liquid aerosolizable material is drawn from each liquid channel 140 to a MEMS die 150 by capillary force.

Two aerosol flow paths 153 are arranged to fluidly communicate with the mouthpiece of the aerosol-generating device. Each aerosol flow path 153 allows thus the generated aerosol to flow from a MEMS die 150 to the mouthpiece. In other words, the aerosol flow paths 153 connect air inlets (not shown) within the aerosol-generating device to the mouthpiece for the passage of air through the aerosol-generating device.

A downstream end of each aerosol flow path 153 forms a nozzle 154. The nozzles 154 and the vaporization surfaces 151 are usually on parallel planes. In other words, each nozzle 154 faces a vaporization surface 151.

Each nozzle 154 can be offset from the vaporization surface 151 or alternatively, the nozzle 154 and the vaporization surface 151 may align in direction one above the other.

When a user draws on the mouthpiece, air is brought into the aerosol flow paths 153 through the air inlets connected to the aerosol flow paths 153 so as to create a pressure change that draws the generated aerosol flow to the mouthpiece as it passes over the vaporization surface 151.

In a setup where each nozzle 154 is offset from a corresponding vaporization surface 151, incoming air through the air inlets can flow sideways along the vaporization surface 151 and is then pulled up by the nozzle 154.

Alternatively, incoming air through the air inlets can flow directly into the aerosol flow path 153 over the vaporization surface 151. The nozzle 154 is jetting either perpendicular to, or in parallel with the airflow of the mouthpiece.

Finally, with regard to any details of the inhalation device as such, in particular with regard to the liquid jet device and the interface towards the container, such as the hollow needle 40 and the septum 38 of the container, the disclosure of the applicant's application entitled “Inhalation device with at least one liquid jet device” is incorporated herein by means of the reference.

1. A container (30) for an inhalation device (10) with at least one liquid jet device (24), such as a micro heater or resistor, the container (30) being adapted to deliver liquid to the liquid jet device (24) and having at least one capillary tube (42) extending from a delivery outlet, and at least one absorbing element (46), such as a piece of foam or a wick, adapted to absorb liquid, the capillary tube (42) and/or the absorbing element (46) extending substantially to a bottom of the container (30) opposite the delivery outlet.

2. The container (30) of claim 1, wherein a volume of the capillary tube (42) corresponds to at least 10 μL or at least 15 μL.

3. The container (30) of claim 1 or 2, wherein an inner diameter of the capillary tube (42) is 1-3 mm.

4. The container (30) of one of the preceding claims, wherein the absorbing element (46) is shorter than the capillary tube (42).

5. The container (30) of one of the preceding claims, wherein the absorbing element (46) is attached to a bottom side end of the capillary tube (42).

6. The container (30) of claim 5, wherein a connecting tube (44) having an inner diameter at least as large as an outer diameter of the capillary tube (42) and the absorbing element (46) is placed radially outwards the capillary tube (42) and the absorbing element (46) and connects these.

7. The container (30) of one of the preceding claims, wherein the container (30) has a cap (32), preferably connected to the container (30) by a thread (36), to which the capillary tube (42) is attached.

8. The container (30) of any one of claims 3 to 7, wherein the capillary tube (42) has a length of 5-30 mm and the absorbing element (46) has a length of 3-30 mm.

9. The container (30) of one of the preceding claims, wherein the capillary tube (42) is made from at least one of Teflon, Polypropylene and Polyethylene.

10. The container (30) of one of the preceding claims, wherein the liquid is a propylene glycol/vegetable glycerine mixture or an oil-based perfume.

11. The container (30) of one of the preceding claims, wherein the absorbing element (46) is disposed within the container (30) and/or wherein the absorbing element (46) and the capillary tube (42) are disposed within a same compartment of the container (30).

12. A combination of at least two containers (30) according to one of the preceding claims to form a cartridge (16) insertable into the inhalation device (10).

13. A combination of at least one container (30) according to one of claims 1 to 11 or the cartridge (16) of claim 12 with an inhalation device (10) having at least one liquid jet device (24), which is preferably in the form of a MEMS.

14. A method of conveying liquid to an inhalation device (10) by means of a combination of a capillary tube (42) and an absorbing element (46), such as a piece of foam or a wick, the method preferably being performed using the container (30) of any one of claims 1 to 11 or the cartridge of claim 12.

15. The method of claim 14, wherein the liquid is delivered to the liquid jet device (24) from at least one replaceable container (30) or cartridge (16).
